(19) United States
(12) Patent Application Publication
(10) Pub. No.: US 2015/0018699 A1
(43) Pub. Date: Jan. 15, 2015

(54) SYSTEM AND METHODS FOR CLOSED-LOOP COCHLEAR IMPLANT

(71) Applicants: The Regents of the University of California, Oakland, CA (US); The Provost, Fellows and Scholars of the College o the Holy and Undivided Trinity of Queen Elizabeth, Dublin (IE)

(72) Inventors: Fan-Gang Zeng, Irvine, CA (US); Myles Mclaughlin, Aliso Viejo, CA (US); Thomas Lu, Long Beach, CA (US)

(21) Appl. No.: 14/445,564

(22) Filed: Jul. 29, 2014

Related U.S. Application Data
(63) Continuation of application No. PCT/US2013/023494, filed on Jan. 28, 2013.
(60) Provisional application No. 61/592,329, filed on Jan. 30, 2012.

(51) Int. Cl. A61N 1/36; A61B 5/00; A61B 5/0488; A61B 5/0402; A61B 5/04; A61B 5/0484

(52) U.S. Cl. CPC A61N 1/36032; A61N 1/36067; A61N 1/36139; A61B 5/04001; A61B 5/0484; A61B 5/0488; A61B 5/0402; A61B 5/4836
USPC 600/509; 607/57; 607/45; 600/554; 600/544; 600/546

(57) ABSTRACT

Embodiments of the present disclosure are directed to systems and methods for a closed-loop cochlear implant. The closed-loop cochlear implant can use at least one extra-cochlear electrode for monitoring auditory evoked potentials from the peripheral and central auditory pathway and stimulating to optimize speech processing. The closed-loop cochlear implant can further use at least one intra-cochlear electrode to stimulate the auditory nerve. Additionally, in some embodiments, the closed-loop cochlear implant can be used to monitor biosignals, such as EMG and ECG.

### STATEMENT REGARDING FEDERALLY SPONSORED R&D

The present disclosure was developed, at least in part, with government support under Grant Nos. DC008858 and DC008369, awarded by the National Institutes of Health. The United States Government may have rights to this disclosure.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND

1. Field

The present disclosure relates to cochlear implant systems and methods, and more particularly to a system and method for a closed-loop cochlear implant for monitoring auditory evoked potentials from the peripheral and central auditory pathway to optimize speech processing.

2. Description of the Related Art

A cochlear implant partially restores hearing in deaf people by electrically stimulating the auditory nerve. To date over 200,000 people worldwide have received a cochlear implant. To assess cochlear implant function and to help fine tune the electrical stimulation parameters used in the speech processing strategy, measurements of the brain's response to the electrical stimulation (auditory evoked potentials) are often employed in clinical practice. Currently, responses from the auditory nerve in cochlear implant subjects may be measured by using stimulating electrodes in the implant as recording electrodes. This makes access to these measurements relatively easy and as a result they are widely used in clinical practice. However, the auditory nerve represents only the most peripheral stage in the auditory neural pathway. To measure responses from more central stages in the auditory, visual or somatosensory neural pathways a separate dedicated evoked potential system must be used which involves attaching extra recording electrodes to the scalp. As a result, responses from the central auditory pathway are infrequently used in a clinic, in spite of the fact that responses from the auditory brainstem and auditory cortex have been shown to be clinically useful in helping to determine the optimal settings for the speech processing strategy.

### SUMMARY

Embodiments of the present disclosure are related to closed-loop cochlear implants and methods for optimizing speech processing using a cochlear implant.

In some embodiments, closed-loop cochlear implant can comprise at least one extra-cochlear electrode configured to detect a neural response, an intra-cochlear electrode configured to stimulate a patients auditory nerve, and a processor coupled to the extra-cochlear electrode and the intra-cochlear electrode. The processor can be configured to monitor the detected neural response and calculate stimulation parameters to improve the neural response. The processor can be further configured to assess the functionality of the visual system or the somatosensory system. In some embodiments, the cochlear implant can further comprise a third extra-cochlear electrode located at an orthogonal placement from the other electrodes.

In some embodiments, the implant can monitor auditory evoked potentials from peripheral and central auditory pathways. The evoked potentials can be selected from the group consisting of compound action potentials, auditory brainstem responses, middle latency responses, auditory steady state responses, and mismatch negativity. An auditory steady state response is the sustained neural response to a sustained modulated auditory stimulus such as a tone or amplitude modulated noise.

In some embodiments, the cochlear implant can comprise a microphone and an antenna, wherein the microphone and antenna communicate using radio frequency signals. In some embodiments, the at least one extra-cochlear electrode is embedded into a patient's skin. In some embodiments, two extra-cochlear electrodes can be used and configured to be sampled at a delay.

In some embodiments, a closed loop system for monitoring biosignals can comprise a first extra-cochlear electrode configured to record a first neural response, a second extra-cochlear electrode configured to record a second neural response, a third extra-cochlear electrode configured to record a third neural response, an intra-cochlear electrode, and a processor coupled to the first, second, and third extra-cochlear electrodes and the intra-cochlear electrode, the processor configured to monitor biosignals from a combination of the intra and extra cochlear electrodes. The monitored biosignals can comprise EEG, EMG, or ECG.

In some embodiments, the third extra-cochlear electrode can be located on a patient's larynx. In some embodiments, the system can be configured to reduce own voice feedback. In some embodiments, the system can be combined with a deep brain stimulator.

In some embodiments, a method for optimizing speech processing in a cochlear implant can comprise implanting an intra-cochlear electrode in a first location in electrical contact with a patient's auditory nerve, implanting an extra-cochlear electrode in a second location, the extra-cochlear electrode configured to monitor the patient's neural pathway responses, monitoring the patient's neural pathway responses through the extra-cochlear electrode, determining simulation parameters from the neural pathway responses configured to provide an optimum neural response, and stimulating the auditory nerve through the intra-cochlear electrode based on the simulation parameters. The method can further comprise optimizing a speech processing strategy. Additionally, the method can further comprise implanting a second extra-cochlear electrode in a third location, wherein the simulation parameters are determined from the neural pathway responses monitored by the extra-cochlear electrode and the second extra-cochlear electrode.

In some embodiments, the cochlear implant can be calibrated without a fitting process. In some embodiments, a dedicated evoked potential system may not be used.

### BRIEF DESCRIPTION OF THE DRAWINGS

The features, objects, and advantages of the present disclosure will become more apparent from the detailed description set forth below when taken in conjunction with the drawings in which like reference characters identify correspondingly throughout and wherein:

FIG. 1 illustrates a graphical representation of a cochlear implant according to one or more embodiments;

FIG. 2 illustrates a simplified block diagram of a cochlear implant according to one or more embodiments;

FIG. 3 illustrates a graphical representation of closed and open loop routes according to one or more embodiments;

FIG. 4 illustrates a forward masking technique according to one or more embodiments of a cochlear implant;

FIG. 5 illustrates extra-cochlear electrode recordings for use in artifact correction according to one or more embodiments;

FIG. 6 illustrates neural responses of a patient according to one or more embodiments of a cochlear implant;

FIG. 7 illustrates quantified neural responses of ECAP, EABR, EAMLR, and loudness according to one or more embodiments of a cochlear implant;

FIG. 8 illustrates collected CEP waveforms in two subjects using scalp electrodes according to one or more embodiments of a cochlear implant;

FIG. 9 illustrates a larynx EMG recorded using standard techniques and using an embodiment of the disclosed cochlear implant recording technique; and

FIG. 10 illustrates a comparison between ECG measurements recorded from an embodiment of the disclosure as compared with standard ECG measurements.

### DETAILED DESCRIPTION OF THE PREFERRED EMBODIMENT

The below disclosure discusses embodiments of cochlear implants and methods to record peripheral and central neural activities, and then adjust the stimulation parameters for improved fitting and performance.

One aspect of the disclosure relates to a system and method for measuring central brain responses in cochlear implant subjects using a cochlear implant. In one embodiment, the extra-cochlear electrode of a cochlear implant (e.g., standard cochlear implant) may be used as a recording electrode and neural responses from the central auditory pathway may be recorded. The extra-cochlear electrode may be used to return the current in monopolar stimulation mode. Another embodiment is directed to recording neural responses from any part of the brain including, but not limited to, the visual system and the somatosensory system. In contrast to previous systems requiring an external dedicated evoked potential system to measure responses in cochlear implant recipients, a method is provided that eliminates the need for such a system and eliminates the laborious process of attaching extra scalp electrodes when collecting these responses. The system and methods as described herein can be employed for a closed-loop cochlear implant system which monitors the neural activity at multiple levels and multiple sites in the brain and uses monitored information to optimize the electrical stimulation parameters and the speech processing strategy. The information from the neural response can also be used to synchronize input from other sensory aids such as a hearing aid, a visual implant or a tactile stimulation aid. The system and methods can also provide access to central brain responses in cochlear implant clinics which do not have an evoked potential recording system and will be a time saving technology for audiologists. The system and methods as described herein can be additionally employed for measuring certain biosignals.

Methods and apparatus are provided for a closed loop cochlear implant. The cochlear implant may be configured to monitor neural pathway response and determine stimulation parameters which provide an optimum neural response. In that fashion, a speech processing strategy may be optimized for individual users.

As used herein, the terms “a” or “an” shall mean one or more than one. The term “plurality” shall mean two or more than two. The term “another” is defined as a second or more. The terms “including” and/or “having” are open ended (e.g., comprising). The term “or” as used herein is to be interpreted as inclusive or meaning anyone or any combination. Therefore, “A, B or C” means “any of the following: A; B; C; A and B; A and C; Band C; A, Band C”. An exception to this definition will occur only when a combination of elements, functions, steps or acts are in some way inherently mutually exclusive.

Reference throughout this document to “one embodiment,” “certain embodiments,” “an embodiment,” or similar term means that a particular feature, structure, or characteristic described in connection with the embodiment is included in at least one embodiment. Thus, the appearances of such phrases in various places throughout this specification are not necessarily all referring to the same embodiment. Furthermore, the particular features, structures, or characteristics may be combined in any suitable manner on one or more embodiments without limitation.

### Cochlear Implants

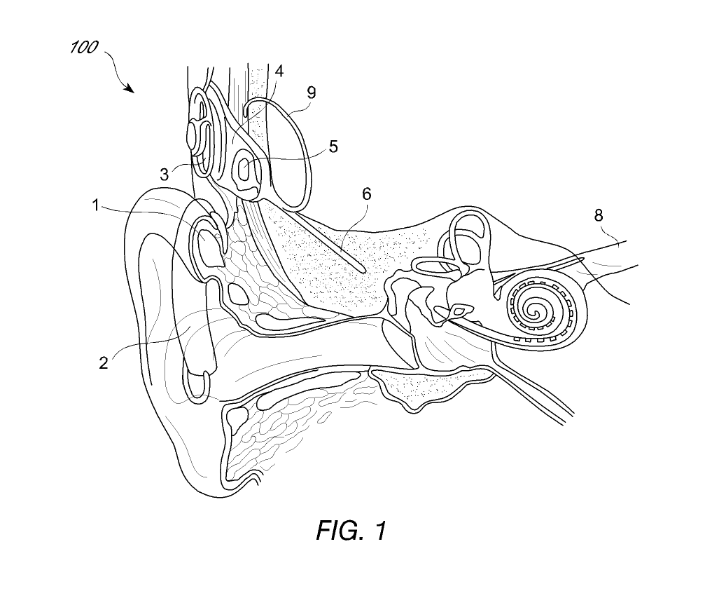

Referring now to the figures, FIG. 1 depicts a cochlear implant according to one or more embodiments. Cochlear implant 100 may be configured to convert sound to electric impulses delivered to the auditory nerve.

FIG. 1 depicts an embodiment of a cochlear implant 100. Cochlear implant 100 can include a behind-the-ear external processor 2 with ear hook 1 and a battery case which can use a microphone to pick up sound, convert the sound into a digital signal, process and encode the digital signal into a radio frequency (RF) signal, and send it to the antenna inside a headpiece 3. Headpiece 3 can be held in place by a magnet attracted to an internal receiver 4 placed under the skin behind the ear. A stimulator 5 contains active electronic circuits that can derive power from the RF signal, decode the signal, convert it into electric currents, and send them along wires 6 threaded into the cochlea. The stimulator 5 may be hermetically sealed. The intra-cochlear electrodes 7 at the end of the wire can stimulate the auditory nerve 8 connected to the central nervous system, where the electrical impulses are interpreted as sound. In a monopolar stimulation mode, electric current can be sent out through intra-cochlear electrodes 7 and returned through one or both of the extra-cochlear electrodes, which can be embedded in the temporalis muscle 9 or attached to the stimulator case 5.

Cochlear implant (CI) 100 may be configured to restore, at least partially, hearing in deaf individuals by electrically stimulating the auditory nerve via an electrode array implanted in the cochlea. External behind-the-ear (BTE) 2 processor can run a speech processing strategy which controls this electrical stimulation. A radio-frequency (RF) link may be employed for two-way communication between the internal receiver and the external BTE processor 2. Accurate control of the electrical stimulation is useful in delivering acoustic information to the auditory nerve 8 which can then be intelligibly interpreted by the brain. The development of processing strategies which could effectively deliver speech information to the brain is one of the elements in the success story of modern CIs.

Typically, when a cochlear implant is switched on for the first time, and at regular intervals thereafter, the electrical stimulation must be carefully adjusted for each individual user by an audiologist. This process is referred to as fitting the cochlear implant and can be a time consuming process for both the audiologist and implantee. Evoked potential measurements provide an objective measure of cochlear implant function which can assist with the fitting process. However, to record more central brain responses, such as auditory brainstem or cortex, a dedicated evoked potential measurement system typically must be used. The dedicated evoked potential measurement system involves attaching scalp electrodes to the subject, and then amplifying and digitizing the signal using dedicated hardware and software. Many cochlear implant clinics do not provide such a system. In addition, preparing the skin and attaching scalp electrodes is a time consuming process. According to one embodiment, auditory brain stem responses can be used to determine threshold and comfort levels for the electric stimulation or to assess more central processes like integration of sounds from both ears. Cortical responses can be useful for predicting more complex functions like speech perception. However, the subject preparation time and the availability of dedicated evoked potential recording systems limit the clinical use of both of these measurements.

According to one embodiment, an evoked potential recording system is provided to record neural activity from any region of the brain. This recorded information can be used, for example, to assess the functionality of the visual system or the somatosensory system.

In one embodiment, neural responses at multiple levels in the neural pathway can be recorded using only the cochlear implant, such as cochlear implant 100. According to another embodiment, cochlear implant 100 may be configured as a closed-loop cochlear implant. Cochlear implant 100 may be configured to monitor neural responses, determine which electrical stimulation parameters give the optimal neural response, and thus, automatically optimize the speech processing strategy for each individual user. Thus, one benefit of cochlear implant 100 may be eliminating the need for a fitting process, which saves time for both the audiologist and implantee. A closed-loop cochlear implant system which monitors neural responses from the somatosensory system could be used to synchronize tactile and auditory stimulation, enhancing the benefit from both devices.

Another benefit of cochlear implant 100 may be that a dedicated evoked potential system is no longer needed to record evoked potential responses in cochlear implant clinics. Rather, employing cochlear implant 100 may allow for cochlear implant clinics to access responses from more central regions of the brain without the need for dedicate evoked potential system and without the laborious process of attaching scalp electrodes. Also, because the extra-cochlear electrode can be embedded in the skin, the recorded responses tend to be much larger and improve signal to noise ratio.

According to one embodiment, two extra-cochlear electrodes, such as electrodes 5 and 9 can be used as recording electrodes, and an intra-cochlear electrode can be used as an indifferent electrode. The neural signal detected by electrodes 5 and 9 may then be sampled at the appropriate delay to capture the desired response. In a particular embodiment using a cochlear implant system with extra dedicated recording electrodes (as described below), the spatial orientation of the electrodes may be used to isolate responses from one particular region of the brain. As with all auditory evoked potential recordings the neural responses may be small compared to the background noise. Therefore, the stimulus may be repeated a number of times and the recorded signal may be averaged. Averaging can cancel the background noise and enhance the auditory neural response which is synchronized to the stimulus. Switching on the amplifier used for the back telemetry can cause an amplifier switch on artifact. In some embodiments, to remove this artifact, the switch on artifact may be recorded alone, without any electric stimulation. As such, this recording contains a switch on artifact only. This switch on artifact may then be subtracted from a normal recording which contains both neural responses and switch on artifact, resulting in the desired neural response.

Cochlear implant 100 may be configured to record evoked potentials from the central brain. In contrast to typical cochlear implants, cochlear implant 100 may include one or more design modifications, hardware and/or software for improving the applicability of the new technique and to allow for closed-loop functionality. Cochlear implant 100 may include a third extra-cochlear electrode dedicated to monitoring neural responses. The third extra-cochlear electrode can be placed at some distance from the intra-cochlear electrodes and other two extra-cochlear electrodes. An orthogonal placement of this third extra-cochlear electrode to the existing electrodes may facilitate the measurement of larger neural response and smaller artifacts. In some embodiments, cochlear implant 100 may include better control over the amplifier, A/D convertor and fitting protocol to allow for lower sampling rates and longer sampling windows than with existing cochlear implants. Thus, a more efficient and flexible collection of evoked potentials may be provided from various stages of the nervous system. Additionally, in some embodiments, cochlear implant 100 may provide bilateral cochlear implantation. As bilateral cochlear implant system, cochlear implant 100 may allow for recording and indifferent electrodes on separate bilateral cochlear implants would allow for greater flexibility in the spatial orientation of electrodes and thus facilitate measurement of larger neural responses and smaller artifacts. According to another embodiment, cochlear implant 100 may include embedded software in the behind-the-ear processor to automatically, and on an ongoing basis, monitor the auditory evoked potentials at multiple levels and multiple sites in the brain. The auditory evoked potentials can then be used to automatically adjust the electrical stimulation parameters for each individual user. For example, responses from the auditory nerve or brainstem can be used to set comfort and threshold levels, auditory brainstem responses can be used to synchronize bilateral devices (e.g., either one cochlear implant on each ear or a cochlear implant on one ear and a hearing aid on the other) and cortical responses can be used to assess more complex functions like speech perception. Responses from the somatosensory system could be used to improve integration with tactile stimulation devices and response from the visual system could be used to improve integration with a visual prosthesis.

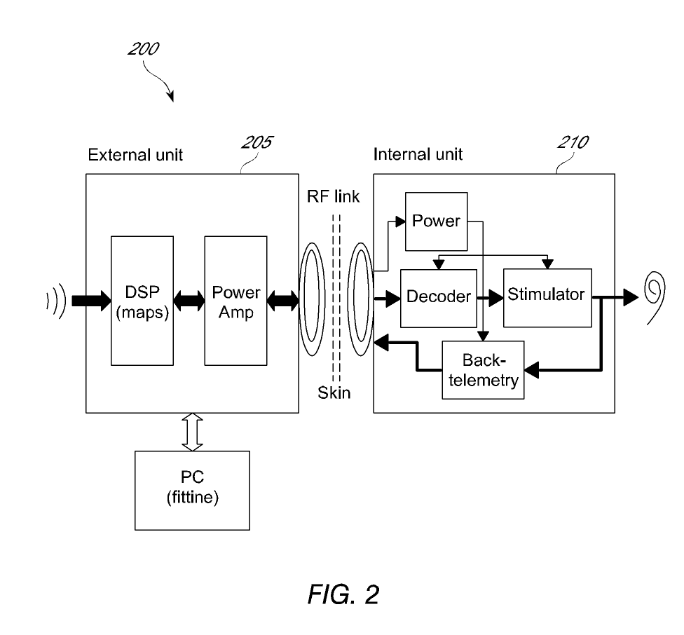

FIG. 2 depicts a simplified block diagram of the cochlear implant of FIG. 1 according to one or more embodiments. Cochlear implant 200 can include external unit 205 and internal unit 210. External unit 205, also known as the speech processor, can include a digital signal processing (DSP) unit, a power amplifier, and an RF transmitter. Internal unit 210 can include a RF receiver and a hermetically sealed stimulator. Because internal unit 210 may have no battery, the stimulator can derive power from the RF signal. The charged up stimulator will then decode the RF bit stream and convert it into electric currents to be delivered to appropriate electrodes.

Cochlear implant 200 may be configured to perform a back telemetry process where the stimulating electrodes in the cochlea (e.g., intra-cochlear electrodes 7) can be used as recording electrodes to capture the response of the auditory nerve (e.g., auditory nerve 8) and provide the response back to the behind the ear processor (e.g., processor 2) and eventually to a PC.

According to one embodiment, cochlear implant 200 may be configured to record central evoked potential responses using only the cochlear implant as a recording device. According to another embodiment, cochlear implant 200 may be configured as closed-loop cochlear implant system which automatically measures neural responses at multiple levels and multiple sites in the brain and uses this information to optimize the speech processing strategy and/or to synchronize stimulation with other sensory aids.

In some embodiments, a single extra-cochlear electrode and a single intra-cochlear electrode can be used for cochlear implant recording. However, these numbers are not limited. In some embodiments, up to about 2, 3, 4, 5, 6, 7, 8, 9, 10, 11, 12, 13, 14, 15, 16, 17, 18, 19, 20, 21, 22, 23, 24, 25, 26, 27, 28, 29, 30 or more intra-cochlear electrodes can be used, with about 1, 2, 3, 4, 5, 6, 7, 8, 9, 10, 11, 12, 13, 14, 15, 16, 17, 18, 19, 20 or more extra cochlear electrodes. In one embodiment, 22 intra-cochlear electrodes are used with 2 extra-cochlear electrodes. Increasing the number of extra-cochlear electrodes can be advantageous for recording a wider range of biosignals, as discussed in more detail below, with improved signal-to-noise ratios and better artifact cancellation.

I. Auditory Pathways

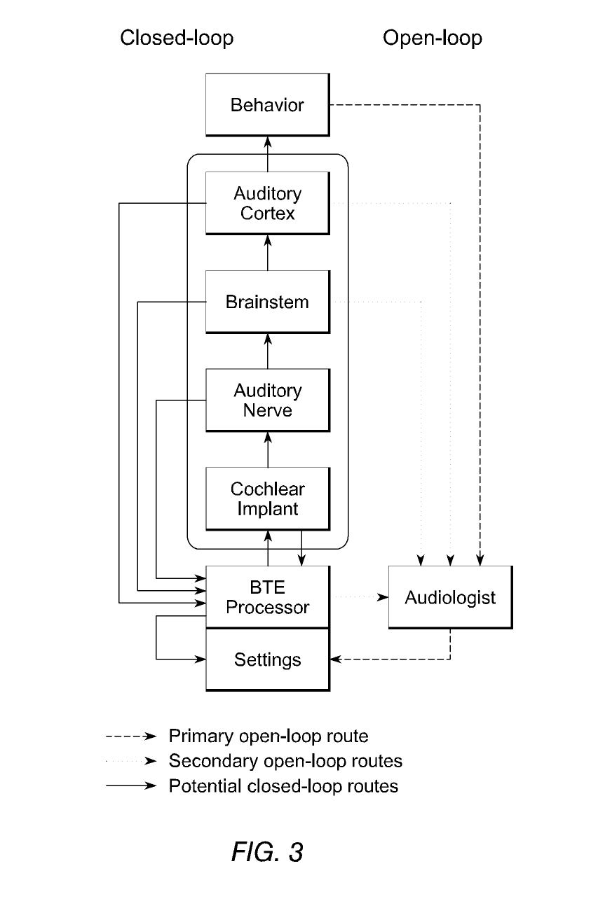

FIG. 3 depicts a graphical representation of closed and open loop routes according to one or more embodiments. The open loop process of ‘fitting’ or ‘mapping’ the cochlear implant (CI) (see Table I for a summary of the different steps) may be carried out by an audiologist and involves carefully selecting the correct speech processing strategy and setting the electrical stimulation parameters for each individual user. Properly fitting the CI is desirable so the recipient can successfully understand speech.

TABLE 1

| | Closed- | |
|---|---|---|
| Fitting Steps | Loop | Technique |
| Assess electrode-neuron | Yes | Impedances checked automatically |
| interface | | |
| Set threshold level | No | Behavior, but ECAP is increasingly |
| | | used in infants |
| Set comfort and loudness | No | Behavior, but ECAP or stapes reflex |
| growth | | are used |
| Discrimination | No | No standard, but MMN could be |
| | | useful |
| Speech Recognition | No | No standard, N100 or later ECEP |
| | | could be useful |

Typically, most of the fitting steps are done in an open-loop system: the audiologist stimulates a CI electrode, elicits a verbal response and accordingly adjusts a setting on the BTE processor (see FIG. 3, primary open-loop route). There are a number of disadvantages associated with this open-loop method. First, it is time consuming for both the audiologist and the CI user. Fitting a CI can last anywhere from ten minutes to a couple of hours, and as the optimal settings for each individual user can change during the first few months of use the fitting process is often repeated. Second, in an open-loop system there is no effective way to determine each user's optimal settings for speech recognition. The stimulation current level that just elicits an auditory percept (threshold or T level) and that which is most comfortable (comfort or C level) are relatively easy to determine behaviorally. However, when post-lingually deafened adults are first fitted with a CI they go through a relearning period where the brain learns to interpret the spectrally-impoverished electrical stimulation delivered by the speech processing strategy as meaningful auditory input. This relearning period can last anywhere for a few weeks to a year or more. Therefore, simply changing speech processing strategies and asking a CI user if it ‘sounds better’ is neither an effective nor efficient way of choosing the correct stimulation parameters as the brain needs time to adjust to the new input. A further disadvantage of an open-loop system is that it requires verbal feedback from the CI user. As CI technology advances it is quickly becoming the standard treatment for children who are born with severe to profound hearing loss in the developed world. A number of studies report and recommend implantation in very young children but obtaining meaningful verbal responses in these children is difficult and sometimes impossible. According to one embodiment, a closed-loop CI, with access to neural responses at multiple levels along the auditory pathway, could perform these tasks automatically and resolve many of these issues.

In fact, the development of the two-way communication between the CI and BTE processor means that the electrodes normally used to stimulate the cochlea can be used as recording electrodes to obtain electrical compound action potentials (ECAP) from the auditory nerve, the first stage in the auditory neural pathway. Scalp electrodes can be used to monitor neural activity further along the auditory pathway in the brainstem or auditory cortex. Therefore, the audiologist does have access to a number of evoked potential (EP) measures of auditory neural activity and, particularly in pediatric populations, these measures are sometimes used to guide the fitting procedure (see FIG. 3, secondary open-loop routes).

Cochlear implants can allow for two-way use of both intra-cochlear and extra-cochlear electrodes (e.g. both stimulation mode and recording mode). This two-way functionality, on both intra- and extra-cochlear electrodes, can be used for a closed-loop cochlear implant. For stimulating functionality, electrodes can be positioned in the cochlea (intra-cochlear) to stimulate the auditory nerve. For the recording functionality, the electrodes can be positioned outside the cochlea, and it is not necessary that any electrodes are positioned in the cochlea. At least two electrodes are needed to record functionality, and these should have some spatial separation; a few centimeters will suffice, but recording will likely improve with more separation.

Embodiments are directed to a method for recording longer latency EPs using only the CI. In addition to the intra-cochlear electrodes used for stimulation, extra-cochlear electrodes of the cochlear implant which are used to return the current in monopolar stimulation mode can also be used to record the neural activity at higher levels in the auditory pathway such as the brain stem and the auditory cortex. Conceptually this closed-loop CI system, with an extended ability to monitor neural activity at multiple stages along the auditory pathway and dynamically adjust the electrical stimulation (see FIG. 3, potential closed-loop routes), could address many of the limitations of the current open-loop system.

Methods of EP recording are provided and discussed below with respect to using an extra-cochlear electrode recording (EER) technique. Section II discusses the standard EP methods used in CI subjects to assess neural activity at different levels in the auditory pathway. In section III, the issues of artifact cancelation and reduction are discussed. In section IV, a discussion of an EER technique is applied and how EER measurements compare with scalp recorded potentials. Section V discusses how the EER technique could be integrated into an embodiment of a closed-loop CI system; how EER technology could be used to monitor neural activity at multiple levels in auditory pathway and used to dynamically adjust the electrical stimulation according to one or more embodiments.

II. Evoked Potentials in the Electrical Stimulated Auditory Pathway

According to one embodiment, EP techniques may be used to measure neural activity at different stages in the electrically stimulated auditory pathway, for example, in the auditory nerve, in the brain stem and in the cortex. For measuring neural activity in the auditory nerve, one electrode can be located in the cochlea and one can be located outside the cochlea positioned anywhere on a patients head. For measuring neural activity in the cortex or brainstem, two electrodes can be used in at least any combination of the following locations on a patient: mastoid, either left or right, nape of neck, top of head (Cz), or forehead.

### A. Auditory Nerve

In acoustic hearing the compound action potential (CAP) represents the sum of auditory nerve activity to an acoustic stimulus. A large portion of the auditory nerve is encased in the dense temporal bone making it difficult to get good quality CAP measurements. In animal studies, CAP measurements are typically performed invasively by placing a ball electrode on the exposed auditory nerve trunk or in the cochlea. In people with a CI, intra-cochlear electrodes can be used to obtain high quality ECAP measurements which are simply not possible in non-implanted people. The main difficulty in recording ECAPs is that the auditory nerve response occurs within 1 ms of onset of electrical stimulation (earlier than for acoustic stimulation) meaning that it overlaps in time with the stimulus artifact. A number of techniques for separating the artifact from ECAP response have been developed and are discussed in section III. CI manufacturers have implemented and automated these methods, making the recording of ECAPs in a clinic relatively straightforward with no additional conventional EP equipment required. The commercialization of these techniques means that ECAPs are the most widely used objective measure by audiologists working with CI patients.

Once the ECAP responses have been separated from the artifact they can be used to help guide the choice of comfort and threshold level. The success of this approach has been limited by intra and inter-subject variability. However, combining ECAP measurements with a limited amount of behavioral data can give a reasonable estimate of comfort and threshold levels across all electrodes. ECAPs are also a useful research tool and can be used to assess the spread of electrical excitation within the cochlea, an issue limiting the success of current CIs.

ECAPs from the auditory nerve represent the first encoding stage in the neural auditory pathway. They do not require the subject to be attentive and can be recorded when the listener is sleeping or sedated. As ECAPs are not affected by muscle activity, they can also be recorded when the subject is moving. The ECAP offers a direct assessment of frequency-specific information (cochlear place), which can be more difficult to assess with scalp recording techniques, and have a relatively large amplitude (˜100 μV). They are easier, compared to cortical and brain stem responses, to interpret in young children or people with developmental disorders (both are patient groups which receive CIs). All these properties make ECAPs a good candidate neural response for estimating threshold and comfort loudness levels.

Further, auditory steady state responses can be measured by embodiments of the cochlear implant. An auditory steady state response is the sustained neural response to a sustained modulated auditory stimulus such as a tone or amplitude modulated noise.

### B. Auditory Brainstem

The next auditory encoding stages occur in the brain stem. The auditory brainstem response (ABR) represents activity from structures in the brainstem, including the auditory nerve, cochlear nucleus, superior olivary complex and inferior colliculus. The maximal latency of the components of the acoustic ABRs are restricted to around 10 ms but electrical ABR (EABR) latencies occur a few milliseconds earlier because electric stimulation bypasses any acoustical mechanical delays present in the middle and inner ear (e.g. the basilar membrane). Because EABRs are more delayed than ECAPS, they are technically easier to separate from the artifact. The EABR can be recorded by placing a scalp electrode on the vertex or forehead and one on the mastoid (although other configurations are possible) and amplifying the potential difference between these two electrodes. A stimulus is typically repeated 2000 to 4000 times, and the EABR is calculated by averaging the recorded epochs.

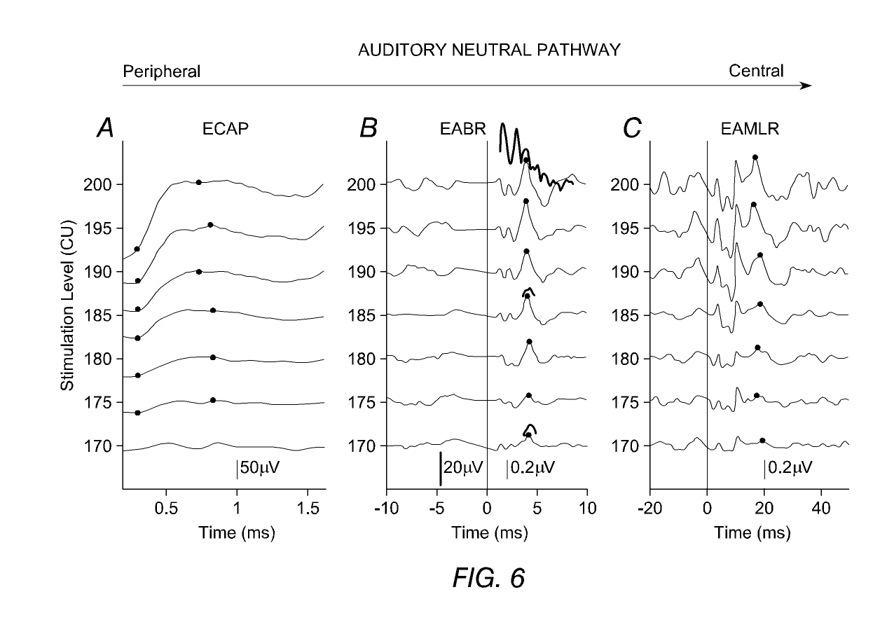

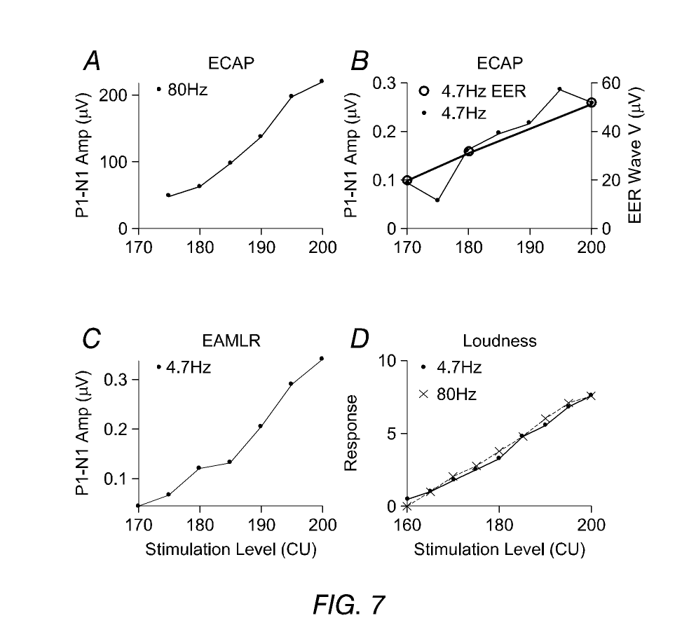

Each peak in the averaged ABR is typically labeled with Waves I through V, with each wave representing activity at a different site in the brain stem. Wave V is the largest component of the EABR and again, because of the lack of acoustic delay, it occurs earlier in CI subjects than in normal hearing (˜4 ms vs. 5.7 ms). It can be used to help predict comfort threshold levels and EABR threshold have been shown to be closer to behavioral thresholds than ECAP thresholds (see FIGS. 6 and 7).

EABRs represent more central processing than ECAPs and as such can be used to study binaural integration. As more and more people receive bilateral CIs, the question of how to optimize bilateral electrical stimulation so that the brain can fully integrate the information from both ears becomes increasingly important. In persons with normal hearing, the binaural interaction component (BIC) can be calculated by subtracting the left monaural and right monaural ABRs from the binaural ABR. It was shown in implanted cats that when a bilateral pair of electrodes stimulates the same frequency region the amplitude of the BIC is largest. It was hoped that this technique could be used to objectively pitch match electrodes in bilateral CI users and thus enhance binaural integration. In some embodiments, focusing on the monaural wave Vs may improve measurements. For example, using the amplitude to objectively loudness balances bilateral CIs or peak latencies to objectively synchronize their timing.

### C. Cortex

It is reasonable to infer that examining CI function at higher levels of auditory processing (i.e., cortex) may provide better relationships with speech perception compared to subcortical responses. In general, early, or “obligatory” cortical potentials (less than 200 ms) represent initial cortical processing and reflect stimulus attributes, while later latency potentials reflect different degrees of processing of the stimulus such as discrimination.

The middle latency response (MLR) is a series of positive-negative waves occurring between 15 and 50 ms. The MLR likely represents the first cortical response that can be recorded using scalp electrodes. Although the MLR varies with stimulus intensity, the relationship with speech perception performance has been poor. In subjects with poor speech perception, larger degrees of ELMR variability have been found. It has been determined that a significant relationship with speech perception using normalized EMLRs as a function of threshold and dynamic range.

The N100, the name of assigned to the negative peak in a cortical evoked potential, is recorded as a negative deflection occurring close to 100 ms and is most often elicited by an onset of a stimulus such as a tone or speech. In adult CI-users, when the N100 is present, it is often similar in morphology to that observed in normal hearing adults although may be reduced in amplitude. The N100 change response, a stimulus that shares acoustic properties with speech, has been shown in a number of studies to relate to speech perception in CI subjects and may provide an index which relates to speech discrimination ability. However, an abnormal “N100” or “deprivation negativity” has been reported only in subjects with especially poor speech perception scores. It has been found that children who had speech perception performance above 90% showed an age appropriate positivity (PI) whereas poor speech performance was associated with a negativity resembling an early N100.

The mismatch negativity (MMN) is a negative deflection that is seen when subtracting the evoked response to frequent stimuli (standard) from responses to infrequent stimuli (deviant). The derived negativity is thought to be related to the subject's ability to discriminate standard and deviants. In the context of CI users, reports have used speech contrasts, tones and CI electrode pairs as stimuli. The general finding with these studies is that a MMN can be recorded in both adult and child CI users. However, the relationship between MMN and speech perception ability is varied. It has been found that MMN to tonal contrasts was related to speech perception performance but MMN to speech contrasts was not. It has been found that good CI performers had a higher probability of having a MMN to speech stimuli than poor performers. A significant relationship was observed with MMN duration and speech perception. Therefore, MMN likely has some predictive ability of speech perception.

III. Artifact Reduction and Cancelation

Two distinct types of artifact exist in CI EP recordings: one results from the stimulation of the electrodes in the cochlea and is referred to here as the stimulation artifact. It is visible as a bipolar spike in the EP recordings which typically last from tens to hundreds of microseconds but can be a few milliseconds in duration (exponential decay of the artifact and amplifier recovery from saturation may extend it). The shape of stimulation artifact is determined by the shape of the stimulation pulse and it reverses when stimulus polarity is reversed. The second artifact results from the RF communication link between the BTE processor and the CI and may be caused by a capacitive coupling between the RF link and the recording leads or electrodes. This may be referred to as the RF artifact, and it is often visible as an elevated pedestal or DC component in the EP. It does not reverse with stimulus polarity reversal. Artifact reduction techniques seek to minimize the size of the artifact before it is recorded, while cancelation techniques seek to remove the artifact after it has been recorded. Both reduction and cancelation strategies are essential and often used in combination to record EPs in CI users.

### A. Artifact Reduction Techniques

One method to reduce both the stimulus and RF artifacts is to increase the spatial distance between recording electrode and CI, for example by placing the recording electrode on the mastoid contralateral to the CL. Other techniques to reduce the RF artifact include spatially separating the recording leads from CI to minimize any current which may be induced and keeping recording leads close together so any induced artifact will be the same in the reference and recording lead and thus be rejected by the differential amplifier. Increasing the temporal separation between the stimulation pulse and recording epoch is another straight forward and extremely effective method of reducing the stimulus artifact. This can be achieved by using low rate stimulation and recording in the gaps between the stimulus pulses. However, this approach is not suitable for recording auditory nerve responses which occur temporally very close to the stimulus. It also is not useful for recording EPs on CI with the clinically relevant pulse rates of 1000 Hz or higher. Finally, stimulus artifact reduction can be achieved by careful design of the stimulus pulse. Tri-phasic pulses can be employed where the final phase of the pulse should cancel or reduce the exponential decay of the artifact.

B. Artifact Cancelation Techniques Artifact template Subtraction

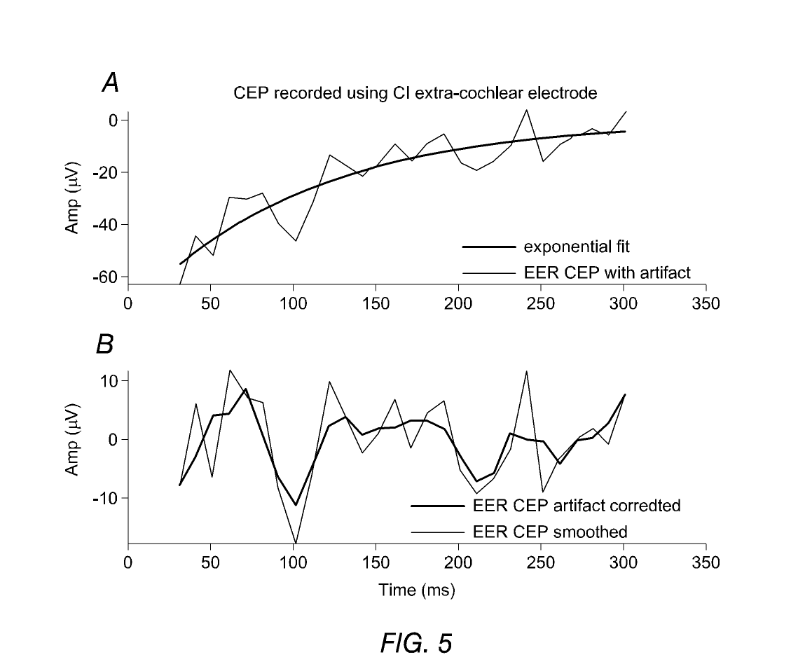

Artifact template subtraction is an effective signal processing technique that can remove stimulus artifact by subtracting a template containing artifact only from the contaminated signal which contains both artifact and neural response. In CIs and other neurotechnology applications, such as deep brain stimulation, the difficulty is to obtain a clean estimate of the stimulus artifact. One method of doing this is to assume the stimulus artifact scales linearly with current level, record a stimulus artifact at a subthreshold current level and then linearly scale it to the required level. In practice, the linearity assumption is not always true due to nonlinearities in tissue conductance and signal amplifier. If an artifact-only template is not available, then a model about the shape of the artifact can be made, for example an exponential decay artifact. By fitting the contaminated response with the assumed artifact function, a template for subtraction can be obtained as depicted in FIG. 5. The main limitation of this technique is that the assumed artifact function may not accurately describe the artifact. The forward masking paradigm below describes a more sophisticated method for estimating the artifact template that does not explicitly assume template linearity or function.

### Forward Masking

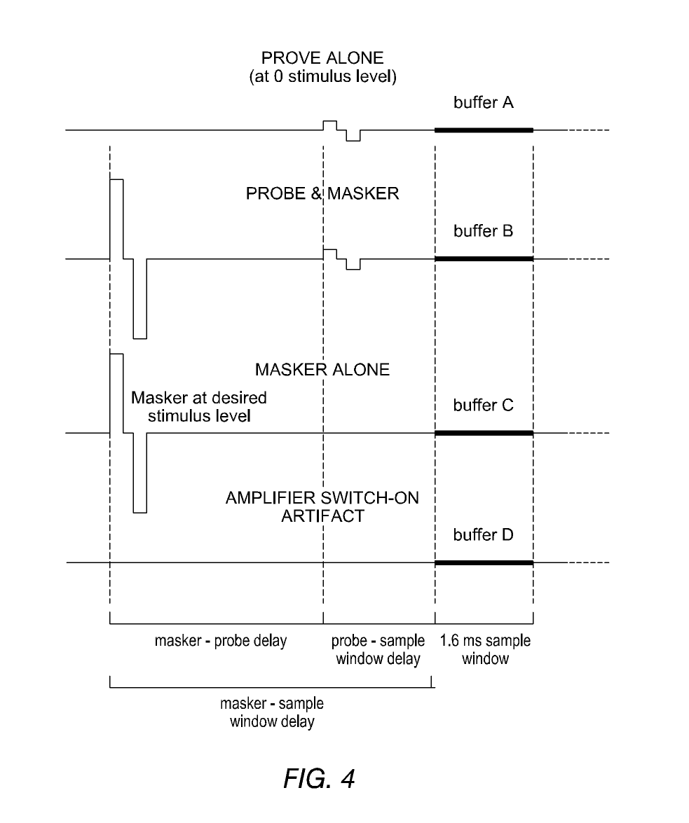

The basic principal of the forward masking technique is shown in FIG. 4. The response to a probe stimulus alone is recorded, which contains both the neural response and the stimulus artifact. The response to probe stimulus that was quickly preceded (<lms) by a masker stimulus is recorded. This contains only the stimulus artifact and no neural response as the forward masker still subjects the auditory nerve to its absolute refractory period. The artifact-only response is then subtracted from the neural response plus stimulus artifact, leaving only the neural response. The complex forward masking algorithm has been implemented and automated by the major CI manufactures, making it relatively easy to apply in practice. However, it can still be a time consuming technique and needs to be carefully set to cleanly remove stimulus artifact.

### Alternating Phase

Biphasic pulses (cathodic followed by anodic) are typically used in CI stimulation. The stimulus pulse can be reversed in polarity so that the anodic pulse comes first. Assuming the stimulus artifact simply reverses in polarity but the neural response will not, then summing the response to two pulses of opposite phase should cancel the artifact and leave twice the neural response. This technique can provide a large reduction in stimulus artifact but it will normally not give complete artifact cancelation due to asymmetries in tissue conductance and the amplifier.

### Independent Component Analysis

Independent component analysis (ICA) is a blind source separation technique that uses higher order statistics to separate independent sources from signals containing linear mixtures of those sources with the condition that these must be more observation points than sources. ICA has been used to separate the stimulus and RF artifacts from ECEP record with multiple scalp electrodes. A limitation of this technique is the need for multiple recording sites and therefore requires a subjective evaluation of presumed artifact scalp topography. ICA is therefore not suitable to the current implementation of EP recordings using embedded cochlear implant hardware described below.

IV. Extra-Cochlear Electrode Recordings

In spite of much research and promising results regarding speech performance predictions, EP techniques measuring neural responses in the brainstem and auditory cortex are not widely used in clinical settings. One reason for this lack of clinical usage is that they require the use of an additional, dedicated EP recording system which takes time to setup and is not usually available in a clinic. This problem, together with the potential applications for a closed-loop CI system, prompted development of a technique to use the extra-cochlear electrodes as recording electrodes for longer latency neural responses without the need for a separate EP recording system. In this section the EER technique is described and EER responses compared with those obtained using scalp electrodes at different levels in the auditory pathway in 2 post-lingually deaf CI subjects using the Nucleus 24 (Cochlear Corporation, Australia). Both subjects were female, aged 74 and 69 years, and were considered good users.

### A. Methods

### Scalp Electrode Recordings and Stimuli

A custom built recording, artifact cancelation and analysis system was used to measure EABRs, EAMLRs and CEPs. Two recording electrodes were placed on each mastoid, a reference electrode on the forehead and a ground on the nape of the neck. All recordings were amplified and digitized using the Medusa system from Tucker-Davis Technologies (Alachua, Fla.) consisting of a preamp and AD converter (RA4PA, 48 dB gain, 25 kHz sampling frequency, 2.2 Hz-7.5 kHz 3 dB frequency response), connected via a fiber optic link to the base station (RAI6BA). During a recording session signals were visualized and averaged online and stored on disk for further offline analysis. All the analysis was performed in Matlab (Mathworks, Natick, Mass.) and all filters were Butterworth 2nd order zero-phase filters. Time epochs and filter settings were: 0-10 ms bandpassed at 100-2000 Hz for EABRs, 0-50 ms bandpassed at 50-400 Hz for EAMLR and 0-500 ms bandpassed at 3-35 Hz at for CEPs.

All stimuli were delivered using the Custom Sound EP software and cochlear programming pod (Cochlear Corporation, Australia). This system sends a trigger pulse with each stimulus which was used to trigger our EP recording system. All stimuli were biphasic (25 us per phase, 7 us interphase gap) pulses delivered in a monopolar mode via an intra-cochlear electrode (16) and returned through MPI, the extra-cochlear electrode embedded in the temporalis muscle. For EABRs and EAMLRs a single pulse was repeated at a rate of 4.7 Hz while for CEPs two stimulus settings were used: 1 pulse repeated at a rate of 1.1 Hz or a 500 ms burst at 900 pulses per second repeated at a rate of 0.5 Hz. EABRs and EAMLRs responses represent the average of 4000 repetitions of pulses with alternating polarity, while CEP responses are 300 pulses with the same polarity. All scalp recorded potentials (EABRs, EAMLRs and CEPs) were inverted before plotting, as is the convention for ABRs.

### Intra-cochlear Electrode Recordings and Stimuli

To record ECAP responses from the auditory nerve, a standard Custom Sound EP implementation of a forward masking paradigm was used. The active electrode for the masker and probe was intra-cochlear electrode 16 and the return electrode was MP1. The active recording electrode was 18 and indifferent electrode was the MP2, the second extra-cochlear electrode located on the implant receiver. For ECAPs the 1 probe pulse (25 μs per phase, 7 μs interphase gap) was repeated at a rate of 80 Hz.

### Extra-cochlear Electrode Recordings and Stimuli

For the EERs of longer latency neural responses the Custom Sound EP implementation of the forward masking paradigm may be adapted. The active electrode for the masker and probe may be an electrode and the return electrode may be another electrode. The active recording electrode may be the extra-cochlear electrode and the indifferent electrode may be the intra-cochlear electrode. A recording window of 1.6 ms in duration was sampled and the delay between the masker and the probe was adjusted to latencies long enough to record EABRs and CEPs (see FIG. 4). Therefore, setting the probe level to 0 and the masker to the desired stimulation level, keeping the delay between the probes and recording window fixed at 55 μs, the delay maybe adjusted between the masker and probe to the desired latency (between 1 and 300 ms) minus 55 μs; of course, the settings of the probe level and delay are not particularly critical and can be set at other levels. The recorded data could then be exported and analyzed (e.g., in Matlab). For the EER recordings the data from buffer C in the forward masking paradigm was used and the amplifier switch on artifact contained in buffer D was subtracted (see FIG. 4). To capture the complete EABR a number of overlapping time windows were sampled between 1 and 8 ms by sequentially shifting the 1.6 ms and then patching these responses together. To capture a CEP using the EER technique the 1.6 ms sampling window was sequentially moved in 10 ms steps from 10 to 300 ms and then plotted the averaged the response within each sample window. The stimuli used to collect the EER responses were matched as closely as possible to those used to collect the scalp electrode responses. The stimulating electrode, return electrode, stimulation level and pulse width used to collect the EER EPs were exactly the same as those used to collect the scalp-electrode EPs. However, the Custom Sound EP software was not designed to record these types of responses, meaning that data collection could take a long time. 1000 repetitions were used for data collection for the EER EABR, and 50 repetitions were used for the data collection of EER ECEP. A repetition rate of 4.7 Hz was used for the EER EABR and for the 1 pulse EER ECEP a repetition rate of 1.1 Hz was used. However, for pulse burst EER CEPs a slightly faster repetition rate and shorter pulse burst was used: 222 ms (instead of 500 ms) burst at 900 pulses per second repeated at a rate of 0.7 Hz (instead of 0.5 Hz). The EER EABRs were inverted before plotting to match the convention of the scalp recorded EABRs but the EER CEPs were not inverted.

### Artifact Cancelation and Reduction

To reduce the artifact in the scalp electrode recordings, recording from the mastoid contralateral to the CI and stimuli with temporal gaps during the desired neural responses period were employed. For the EABR and EAMLR recordings, pulses with alternating polarity were used. This reduced the artifact but did not completely remove it. A linear interpolation over a 2 ms period surrounding each pulse was employed to remove the stimulus spike artifact. After this an exponential decay artifact lasting from around 0 to 5 ms still remained. To remove this, a sub-threshold recording was fitted, which contained only artifact, with an exponential function. Finally, the exponential function was linearly scaled to fit each recording and then subtract this scaled exponential from the recording to leave the neural responses.

For the CEP recordings, the alternating polarity pulses were not used as subjects reported differences in loudness to pulse burst with different polarity. To remove the stimulus artifact from the baseline, a 3 ms window around each stimulus artifact was simply linearly interpolated. A slowly decaying exponential artifact was also present in both the scalp electrode and EER CEPs (see FIG. SA). To obtain an artifact template, the recordings were fitted with an exponential function and then the template was subtracted to leave the neural response (see FIG. 5B). The EER CEPs were then smoothed using a 3 point running average.

### B. Results

### Amplitude Growth Functions

The auditory system responded to electrical stimuli at different amplitudes. FIG. 6 traces the neural response in one CI subject to electrical stimulation on the same electrode, with closely matched stimuli, as a function of stimulation levels (170 to 200 clinical units, CU), all the way through the auditory pathway from the auditory nerve (ECAP) to the brainstem (EABR) and the cortex (EMLR). Generally and as expected, neural response amplitudes increased with stimulation levels.

Plot A of FIG. 6 shows the ECAP responses recorded using the standard forward masking paradigm implemented in Custom Sound EP software. N1s and P1s for each response are marked with dots. The amplitude of N1-P1 is clearly related to the stimulation level. The maximal N1-P1 response occurs at 200 CU and has disappeared at 170 CU.

The scalp EABR responses (thin lines, Plot B of FIG. 6) using the same stimulating electrode in the same subject are also dependent on stimulation levels. The amplitude of wave V (at ˜4 ms) increases linearly with increasing stimulation levels. EABR responses obtained using the EER technique (thick line, Plot B of FIG. 6) for three different levels also change similarly with stimulation level. As the Custom Sound EP software was not optimized to record these types of responses, data collection took a long time. The EER EABR at 200 CU from 1 to 8 ms took over 2 hours to collect. Therefore, the time epoch was restricted, the number or repetitions limited to 1000 and data collected at the wave V peak at 185 and 170 CU stimulation levels. There is a reasonable match between the timing of wave V from the scalp electrode recording and that obtained using the EER technique. The polarity of the scalp recorded wave V also matches with the polarity of the EER wave V. However, the waveforms shown have been scaled to approximately match in amplitude. It should be noted that the EER EABR amplitudes are an order of magnitude greater than the scalp electrode EABR (see vertical scale, Plot D of FIG. 7), probably because of reduced impedance due to the extra-cochlear electrode's implanted location and its closer proximity to the neural generator. The section of the EER EABR waveform preceding wave V appears to be elevated when compared to the scalp recorded EABR. This is probably partly due to the stimulation and RF artifacts. Since the return electrode is very close to the auditory nerve, a larger wave II may occur which would contribute to the elevated levels preceding wave V.

Plot C of FIG. 6 shows the scalp-recorded EAMLR recordings in the same subject with the same stimulation parameters as in Plot A of FIG. 6. Here too, the EAMLR shows a strong dependence on stimulation level. Potentially, it would be possible to capture these responses by moving the sample window to longer latencies. The dots on Plot C of FIG. 6 mark the Na peak (negativity occurring around 20 ms) of the EAMLR.

The amplitudes of ECAP, EABR, and EMLR responses were quantified and plotted as a function of stimulation level (Plots A-C of FIG. 7). The plots demonstrate a nearly linear relationship between the amplitude of the measured neural responses and stimulation levels (in current units). FIG. 7B clearly shows that the EABR also decreases linearly with decreasing stimulation levels for both scalp and EER methods.

To provide behavioral assessment for the stimulation levels used, the subject was asked to rate the loudness of the stimuli on a scale of 0 to 10 (0=no Sound, 1=barely audible, 6=most comfortable, 10=extremely loud). Plot D of FIG. 7 shows that the perceived loudness increased with stimulation level. Note that the behavioral response was also linear but the thresholds were lower than the ECAP threshold, as the subject could hear stimuli that produced no observable responses in the ECAP. Since the neural responses were also linearly related to stimulation levels, it follows mathematically that the objectively measured neural responses are also linearly related to subjective loudness.

### Lon Latency Cortical Evoked Potentials

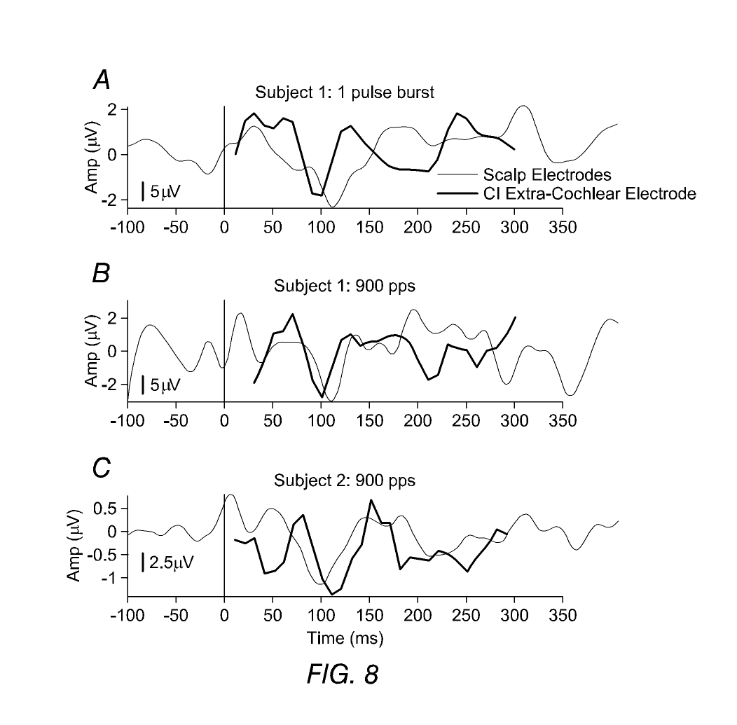

As described, in some embodiments it is possible to record long latency cortical evoked potentials using just the CI and without any dedicated EP equipment. FIG. 8 depicts collected CEP waveforms in two subjects using scalp electrodes. The waveforms were noisy but a clear N100 component was visible in all three recording (panels A-C). The recordings were not corrected for for eye movement artifact by placing an electrode near the eye, but the subject simply closed their eyes during the recordings while remaining alert. This means that the recordings were likely contaminated with eye movement or muscle artifacts and increased alpha-wave activity (˜10 Hz) due to eye closure. However, this also made the scalp-recorded CEPs more comparable to the EER CEP data as it would be difficult to correct for eye movements using the EER technique. EER CEP data was collected using the same stimulus (thick lines on each panel): panel A is 1 pulse repeated every 1.1 Hz, panels Band C are a 500 ms 450 pulse burst repeated every 0.5 Hz. The EER CEP waveforms are also noisy but there is a clear N100 component which aligns reasonably in time with the scalp electrode N100. The amplitudes of the EER CEP waveforms are about an order of magnitude greater than scalp electrode CEPs (note the different scale bars). The polarity of the EER N100 is opposite to that of the scalp recorded N100 (in FIG. 8, for ease of comparison, the scalp recorded waveform has been inverted but the EER waveform has not).

V. Discussion

By using the extra-cochlear electrode in a CI as a recording electrode, it is possible to record longer latency neural responses and that the timings of these responses are in general agreement with auditory brainstem and auditory cortex responses recorded with scalp electrodes. In the past, these higher level responses were only accessible using a dedicated EP system. Now, by using the EER technique, they can be measured using only the CL. The EER technique represents a completely new application for cochlear implants and has an enormous potential for improving implant fitting and performance.

### A. Neural Generators

The response latencies and waveform morphology suggest that an N100 and EABR can be recorded using the present electrodes in the CL The scalp recorded N100 response is made up of multiple generators, but two predominant waveforms are usually seen. First, a vertically orientated dipole arising from auditory cortex that can be seen as negativity near 100 ms recorded at the vertex that is optimally recorded using vertically orientated electrodes. Second, a radially orientated dipole that is observed over temporal electrode recording sites that is optimally recorded using laterally orientated electrodes. The EER electrode “montage” is sensitive to both vertical and radial sources, however, given that CI case is located above (˜10 cm) and slightly lateral (˜2 cm) to the CI intracochlear electrodes, the electrode configuration “axis” will be more sensitive to vertical dipoles. This may explain the difference in polarities of the N100s (on FIG. 8 the scalp recorded waveform has been inverted but the EER waveform has not). Therefore, the EER N100 (FIGS. 5 and 8) likely represents a CI-recorded N100. The EER EABR showed large amplitude waves I and II compared to wave V, a pattern not typically seen with scalp recorded ABRs. Because the generators of wave I and II are likely auditory nerve, the extreme closeness, and reduced impedance of the intra-cochlear electrode likely contribute to their large amplitude.

### B. Applications for the EER Technique

Advances in CI technology and performance have steadily relaxed the criteria for implantation over the years. As a result, the number of people receiving CIs has been steadily increasing across both the developed and developing countries, reaching 200,000 with about half of them being children. Thus, determining beneficial methods and means for fitting a CI efficiently and optimally has become an unmet need. The ECAP technique is regularly used by audiologists to determine threshold and comfort levels. One reason for the preference of the ECAPs over other EPs is that the ECAPS can be recorded using only the CI without the need for a dedicated and often expensive EP measurement system. The other reason is that even when such a system is available, it is a time consuming process for the audiologist due to subject preparation time and cooperation. The development of the EER technique means that this may no longer be an issue. With dedicated software EABRs, EAMLRs and CEPs could be made easily accessible to the audiologist using only the CI as a measurement device. The EER technique could be a vital step towards bringing metrics which access higher level neural responses into general clinical practice; it would not only save time for the audiologist but may also improve CI fitting and performance.

In FIG. 3, a number of routes are depicted along which a closed-loop CI could operate. Auditory nerve response metrics were already accessible via the CI; however, responses from the brainstem and cortex were not. The disclosure demonstrates embodiments of a CI through closure of this part of the loop with innovative use of the existing commercial CI, although some design changes could improve the applicability of the technique as discussed below. A closed-loop CI may be provided which accesses neural responses from multiple levels in the auditory neural pathway. First, the ECAP and EABR responses could be used in a closed-loop CI to automatically set the comfort and threshold levels, eliminating a tedious job for the audiologist. Second, longer latency responses can be used to measure suprathreshold discrimination and recognition tasks, which are not available or used in present clinical settings. For example, the MMN measure could be used to eliminate redundant electrodes that potentially decrease the implant performance. The cortical responses could be used to dynamically adjust the speech processing strategy by tracking corresponding resulting changes in responses.

The ability to track these responses at regular intervals over a long period of time would be a particularly useful application for a closed-loop CI, given the long language learning (pre-lingually implanted) or relearning (post-lingually implanted) period which most CI users go through. As a result, the EER technique can be used as a powerful built-in research tool to study brain maturity and plasticity.

### C. Additional Design Considerations

A number of design modifications are provided for a cochlear implant, both hardware and software related, for improving the closed-loop CI performance. First, a third extra-cochlear electrode dedicated to monitoring neural responses could be placed at some distance from the intra-cochlear electrodes and other two extra-cochlear electrodes. In some embodiments, the intra-cochlear electrode can be moved outside of the cochlea. An orthogonal placement of this third extra-cochlear electrode to the existing ones may facilitate the measurement of larger neural response and smaller artifacts. Second, better control over the amplifier, A/D convertor and fitting protocol would allow for lower sampling rates and longer sampling windows than the existing CIs, and thus enable more efficient and flexible collection of evoked potentials from various stages of the nervous system. Third, software on the BTE processor could be used to track neural responses over time and wireless technology in the BTE processor could send and log this data on a personal wireless device such as a smart phone, where it could be remotely accessible by the audiologist. These design changes and considerations can and should be implemented in not only next-generation cochlear implants but also other neural prostheses and stimulators such as retinal implants and deep brain stimulation devices.

VI. Recording Biosignals

In addition to the above described measurements, embodiments of the disclosed CI could be used to record other biosignals, such as, for example, recording electroencephalograms (EEG), electromyograms (EMG), and electrocardiograms (ECG).

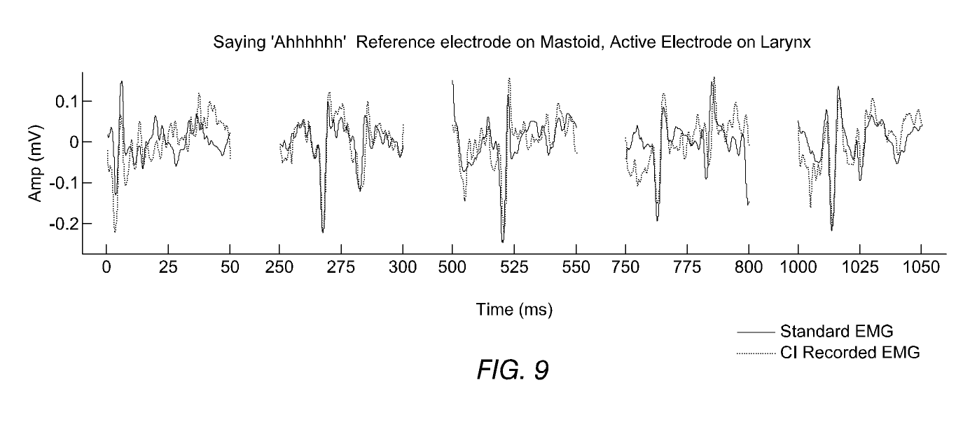

The standard position of the extra-cochlear electrode is in a temporal muscle. As discussed above, using an extra-cochlear electrode in this standard position and an intra-cochlear electrode allows the recording of EEG activity. However, placing an additional extra-cochlear electrode at a more distant location, such as, for example, on the larynx, can allow for an accurate recording of EMG and EEG activity. FIG. 9 shows a larynx EMG recorded during vocalization in one subject using standard techniques and recorded using the CI recording technology. As shown, there is good agreement between the timing and shape of the signals recorded using both methodologies.

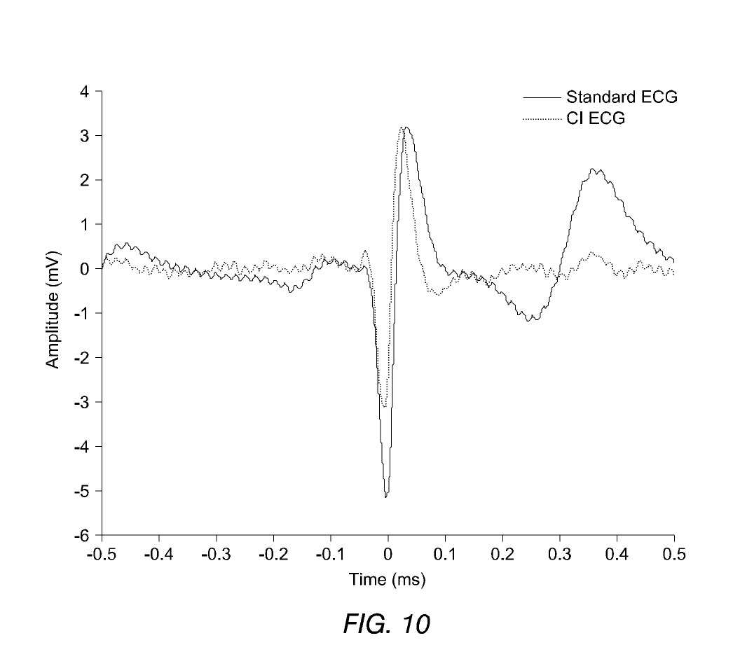

Further, the same electrode configuration can also allow for the recording of ECG activity with a CI. FIG. 10 illustrates a comparison of ECG measurements taken using an embodiment of the disclosed CI with the measurements of ECG using standard ECG measuring equipment. As shown, the CI measurements track extremely well with the standard ECG equipment, illustrating how embodiments of the disclosed CI could be used to accurately measure ECG levels. The ECG recording technique could be used to report heart rate information to neural implant users; useful during sports activities or for users with known health issues. Monitoring of the ECG signal could also provide users with an early warning about potential cardiac problems.

The CI biosignal recording technique could be used to detect when a CI user is speaking. This information could be used in the implant to reduce a patient's own voice feedback and improve noise cancelation algorithms by removing the speakers own voice from the mix. The EMG monitoring technology could be applied to other neural implants, such as deep brain stimulators (DBS). Motion disorders, such as Parkinson's disease are regularly treated with a DBS, which operates in an open-loop system. The EMG recording technique would allow for the design of a closed-loop DBS which monitors muscle activity and uses this information to actively control the amount of neural stimulation.

Although the foregoing description has shown, described, and pointed out the fundamental novel features of the present teachings, it will be understood that various omissions, substitutions, and changes in the form of the detail of the apparatus as illustrated, as well as the uses thereof, may be made by those skilled in the art, without departing from the scope of the present teachings. Consequently, the scope of the present teachings should not be limited to the foregoing discussion, but should be defined by the appended claims.

1. A closed-loop cochlear implant, comprising:
at least one extra-cochlear electrode configured to detect a neural response;
an intra-cochlear electrode configured to stimulate a patient's auditory nerve; and
a processor coupled to the extra-cochlear electrode and the intra-cochlear electrode, the processor configured to monitor the detected neural response and calculate stimulation parameters to improve the neural response, the processor further configured to assess at least one of a functionality of a visual system and a somatosensory system.

2. The cochlear implant of claim 1, wherein the implant monitors auditory evoked potentials from peripheral and central auditory pathways.

3. The cochlear implant of claim 2, wherein the evoked potentials are selected from the group consisting of compound action potentials, auditory brainstem responses, middle latency responses, auditory steady state responses, and mismatch negativity.

4. The cochlear implant of claim 1, further comprising a microphone and an antenna, wherein the microphone and antenna communicate using radio frequency signals.

5. The cochlear implant of claim 1, wherein the at least one extra-cochlear electrode is embedded into a patient's skin.

6. The cochlear implant of claim 1, further comprising two extra-cochlear electrodes configured to be sampled at a delay.

7. The cochlear implant of claim 6, further comprising a third extra-cochlear electrode located at an orthogonal placement from the other electrodes.

8. (canceled)

9. A closed loop system for monitoring biosignals, comprising:
a first extra-cochlear electrode configured to record a first neural response;
a second extra-cochlear electrode configured to record a second neural response;
a third extra-cochlear electrode configured to record a third neural response;
an intra-cochlear electrode; and
a processor coupled to the first, second, and third extra-cochlear electrodes and the intra-cochlear electrode, the processor configured to monitor biosignals from a combination of the intra and extra cochlear electrodes.

10. The system of claim 9, wherein the biosignals comprise EEG, EMG or ECG.

11. The system of claim 9, wherein the third extra-cochlear electrode is located on a patient's larynx.

12. The system of claim 9, wherein the system is configured to reduce own voice feedback.

13. The system of claim 9, wherein the system is combined with a deep brain stimulator.

14. A method for optimizing speech processing in a cochlear implant in a patient, the method comprising:
implanting an intra-cochlear electrode in a first location in electrical contact with the patient's auditory nerve;
implanting an extra-cochlear electrode in a second location, the extra-cochlear electrode configured to monitor a neural pathway response in the patient;
monitoring the patient's neural pathway response through the extra-cochlear electrode;
determining simulation parameters from the neural pathway response configured to provide an optimum neural response; and
stimulating the auditory nerve through the intra-cochlear electrode based on the simulation parameters.

15. The method of claim 14, wherein the cochlear implant is calibrated without a fitting process.

16. The method of claim 14, wherein a dedicated evoked potential system is not used.

17. The method of claim 14, further comprising optimizing a speech processing strategy.

18. The method of claim 14, further comprising implanting a second extra-cochlear electrode in a third location, wherein the simulation parameters are determined from the neural pathway response monitored by the extra-cochlear electrode and the second extra-cochlear electrode.
